(19) United States
(12) Patent Application Publication
(10) Pub. No.: US 2017/0128016 A1
(43) Pub. Date: May 11, 2017

(54) EYE PRESSURE DETERMINATION FROM CONTACT WITH AN EYELID USING A MOBILE DEVICE

(71) Applicant: Eye Labs, LLC, Mountain View, CA (US)

(72) Inventor: Sina Fateh, Mountain View, CA (US)

(21) Appl. No.: 14/936,391

(22) Filed: Nov. 9, 2015

(51) Int. Cl. A61B 5/00; A61B 3/16; A61B 5/11

(52) U.S. Cl. CPC A61B 5/6898; A61B 5/1103; A61B 5/7278; A61B 5/7267; A61B 5/7221; A61B 5/7282; A61B 5/0022; A61B 5/7475; A61B 5/742; A61B 3/165; A61B 5/486; A61B 2560/0462; A61B 2562/0247; A61B 2560/0475

(57) ABSTRACT

This application is related to a system for measuring eye pressure and related methods. In some embodiments, the system includes a pressure sensor and a processor. The pressure sensor takes a measurement from a contact with an eyelid. The processor includes an engine that is built from a set of items, each comprising a list of user attribute values, the eye pressure measured by the pressure sensor, and the actual eye pressure level, and can estimate the actual eye pressure level and normalized eye pressure level. Given a new measurement of a user's eye pressure made by the pressure sensor, the processor identifies the user attribute values for the user and runs the engine to estimate the actual eye pressure level and the normalized eye pressure level.

### TECHNICAL FIELD

The present application is related to eye pressure measurement and estimation, and more specifically to methods and systems that determine eye pressures from contact with an eyelid with a mobile device.

### BACKGROUND

High eye pressure can cause glaucoma and permanent vision loss in some individuals. Various types of devices are available for measuring eye pressure. Almost all of them are specialized devices typically operated by an eye doctor in the doctor's office. Many of them require direct contact with the eye, which is associated with sanitation and comfort issues.

### BRIEF DESCRIPTION OF THE DRAWINGS

Various embodiments are disclosed in the following detailed description and accompanying drawings.

FIG. 1 illustrates an example environment diagram in which the system disclosed in the application operates.

FIG. 2 illustrates an example list of user attributes or factors affecting eye pressure.

FIG. 3 illustrates an example process performed by the server for estimating an eye pressure level.

FIG. 4 illustrates an example process performed by the client device for estimating an eye pressure level.

FIG. 5 contains a high-level block diagram showing an example architecture of a computer, which may represent any electronic device, such as a mobile device or a server, including any node within a cloud service as described herein, such as may implement the operations described above.

### DETAILED DESCRIPTION

This application is related to a system and related methods for measuring eye pressure. In some embodiments, the system includes a pressure sensor and a processor. The pressure sensor takes a measurement from a contact with an eyelid. The processor includes an engine that is built from a set of items, each comprising a list of user attribute values, the eye pressure measured by the pressure sensor, and the actual eye pressure level, and can estimate an actual eye pressure level. The user attributes include those that can account for the effect of an eyelid, such as the thickness or resilience of the eyelid. The user attributes also include those that can account for transitory or seasonal effects of environment and personal factors, such as the removal of contact lenses or exercise. By calibrating the measurement data based on the user attribute values using machine learning techniques, the engine can estimate the actual or normalized eye pressure level. Given a new measurement of a user's eye pressure made using the pressure sensor, the processor identifies the user attribute values for the user and runs the engine to estimate the actual or normalized eye pressure level.

Through the use of the system, a user can measure the eye pressure essentially anywhere and anytime. The user does not need to travel to an eye doctor's office, nor does the user need to acquire access to any specialized, delicate, or costly device. However, the user and the eye doctor can receive the measurement result and take actions right away for treatment, prevention, and other purposes. In addition, the user's eyes do not need to receive eye drops or any other external solution for sanitary, anesthetic, or other purposes. In fact, the user's eyes do not experience any particular discomfort from a light press of a regular surface on the eyelid. Furthermore, the user receives an abundance of information, such as a value for the current eye pressure, a value for a normalized eye pressure calibrated on multiple dimensions, and information explaining the deviation of the current eye pressure from the normalized eye pressure.

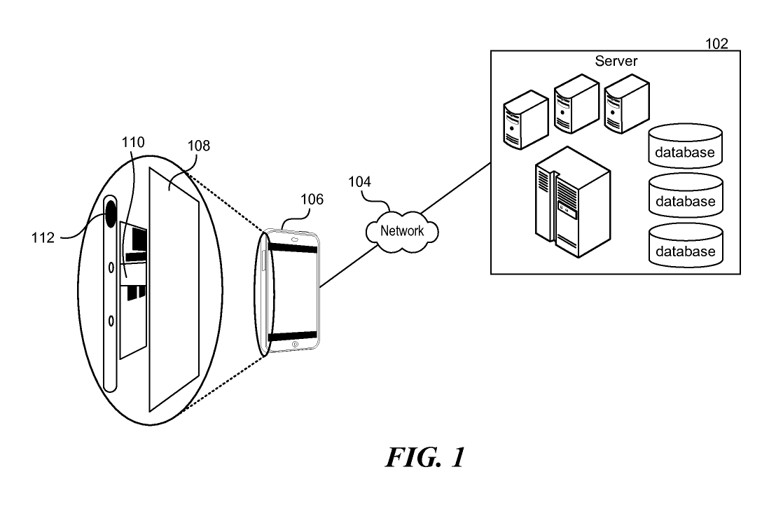

FIG. 1 illustrates an example environment diagram in which the system disclosed in the application operates. In some embodiments, the system includes a server 102 comprising one or more processors and storage devices. The server has sufficient capabilities for large-scale data processing, storage, communication, and so on. The server 102 can be implemented by a cloud-computing platform, a server farm, etc. The system can also include a client device 106 that communicates with the server 102 across a network 104, such as the Internet or a cellular network. The client device 106 can be implemented by a mobile device, such as a cellular phone or a wearable device.

In some embodiments, the client device 106 includes a processor. The client device 106 also includes a display screen 108 that is connected (physically or through a computer network) with the rest of the client device 106. In some embodiments, the processor displays a graphical user interface (GUI) on the display screen 108. The processor receives basic data regarding a user through the GUI. The basic data can include the user's medical history, family history, contact information, various preferences, etc. The medical history can indicate previous diagnosis and treatment of the user's eyes. The family history can include the medical history of family members. The contact information can indicate how to reach eye doctor or personal emergency contacts. The preferences can indicate what types of information to receive or send, when to receive or send such information, how to share such information, etc. The processor can save the basic data locally or send the basic data to the server 102 for further processing and storage. For example, based on the basic data, the system can automatically send periodic reminders to measure eye pressure, or it can notify the user's physician and significant others when the user's eye pressure level exceeds a predetermined threshold.

In some embodiments, the client device 106 includes a touch-sensitive surface, which can coincide with the display screen 108, and a connected pressure sensor 110 that takes a pressure measurement when the surface comes in contact with an object, such as an eyelid of a user. The eyelid can be closed, fully covering the eye, which makes a large area available for contact, or the eyelid can be somewhat open, partially covering the eye, which makes a small area available for contact. The client device 106 instructs the user on how to handle the client device 106 to allow proper pressure measurement. The client device 106 can start communicating with the user when the display screen 108 detects a contact with an object, when the user makes an explicit a request for measuring the eye pressure, etc. The client device 106 can further determine whether the object is human skin (e.g., based on the temperature) or whether the object is an eyelid (e.g., based on the area of contact). The client device 106 can guide the user on how hard or for how long to press the display screen 108 against the eyelid, generally depending on when pressure sensor 112 can detect a stable pressure, using speech, sound, vibration, etc. This client setup can be implemented based on existing techniques, such as one that allows a pressure sensor to be embedded underneath the screen of a cellular phone. Alternatively, the client device 106 can include a pressure sensor 112 that acts as an air puffer, which takes a pressure measurement when the air pulse it transmits applanates the eyelid. In this case, the client device 106 can similarly instruct the user on how to handle the client device 106 to enable proper pressure measurement. For example, the client device 106 can guide the user on at what distance or for how long to hold the pressure sensor 112 near the eyelid. It is to be appreciated by those skilled in the art that the pressure sensor can generally be implemented by modifying existing techniques to require no direct eye contact. In order to use the measured eye pressure to estimate the actual eye pressure level, additional information is needed regarding the user operating the client device.

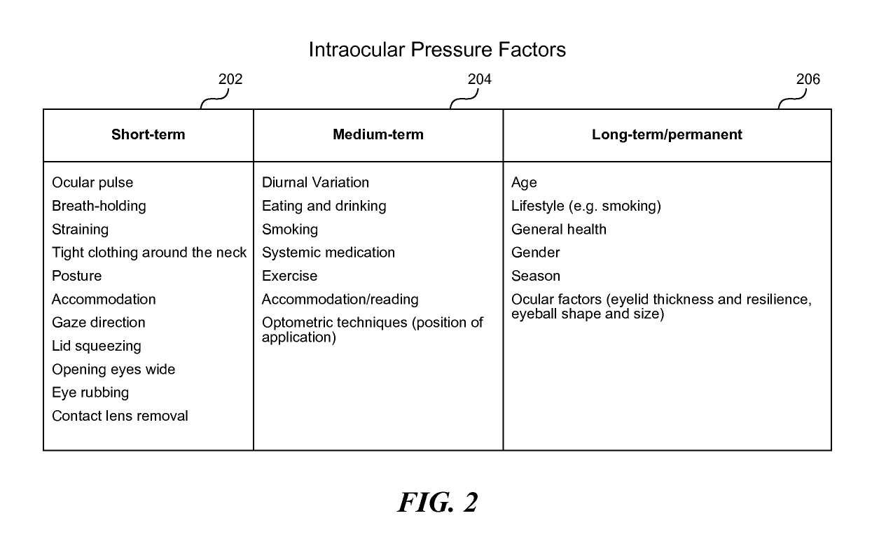

FIG. 2 illustrates an example list of user attributes or factors affecting eye pressure. The user attributes generally include those that account for the effect of an eyelid, including various ocular factors that are directly related to the eyelid and other generally long-term factors 206 that can generally affect the condition of the eyelid, such as age. The user attributes also include those that account for the transitory or seasonal effects of environmental or personal factors. These user attributes can roughly be classified into two categories: short-term factors 202 and medium-term factors 204, although some of the long-term factors can also be used for normalization purposes. Regarding the short-term factors and effects 202, holding the breath or wearing tight clothes often increase the eye pressure, while removing contact lens often tends to reduce the eye pressure, for example. Regarding the medium-term factors and effects 204, recent exercise and anti-hypertensive medication tend to reduce the eye pressure, while tobacco use and large-volume fluid intake tend to increase the eye pressure, for example. Furthermore, the user attributes can include those corresponding to eye diseases, such as glaucoma.

In some embodiments, the processor receives user attribute values from the user through the GUI. The user can supply values for one or more of those user attributes. The values of those user attributes for which the user does not supply values can be supplied by the user's eye doctor, attributed from the values of the other user attributes using known imputation techniques, and so on. The processor saves the user input for possible reuse and transmits the user input, together with the eye pressure measurement, to the server 102 for processing and possibly storage. Subsequently, the processor receives an estimated eye pressure level and other data from the server 102 and displays the received data to the user.

In some embodiments, the server 102 builds an engine for estimating an eye pressure level from a set of items, each comprising a list of user attribute values, the eye pressure measured by the pressure sensor, and the actual eye pressure level, and can estimate the actual eye pressure level. The set of items can be supplied by one or more eye doctors from their own medical practices through a specific user interface. The server can also gather the items from online sources. Using machine learning techniques, the server can build the engine using a subset of the set of items as the training set and a subset of the set of items as the test set. The engine takes a list of user attribute values and a measurement by the pressure sensor of the client device as input and produces an estimate of the actual pressure level as output. Examples of machine learning techniques include linear regression, decision trees, neural networks, etc.

In some embodiments, by accounting for the effect of the eyelid, the server can estimate the eye pressure level even if the pressure sensor takes the measure through the eyelid. Specifically, by training the engine over the set of user attributes that account for the effect of the eyelid, the server can determine the effect of the eyelid from data for other users having similar values in these user attributes and therefore estimate the normalized eye pressure level. It is also possible for the server to train the engine over the entire set of user attributes but only based on those items with normal values for the user attributes that do not account for the effect of the eyelid. By accounting for the transitory or seasonal effects of environmental or personal factors, the server can determine the actual eye pressure level and further focus on the health condition of the eyes. Specifically, by training the engine additionally over the set of user attributes that account for the transitory or seasonal effects (thus the full set), the server can determine those effects from data for other users having similar values in these user attributes and therefore estimate the actual eye pressure level. Therefore, the server can build a two-tier engine, where the first tier is aimed at eliminating the effect of an eyelid, and the second tier is aimed at reproducing the transitory and seasonal effects.

In some embodiments, in addition to an estimate of the actual eye pressure level and an estimate of the normalized eye pressure level, the server 102 generates related data to help the user better understand the computation or the significance of the estimates. The related data can indicate which factors are used in accounting for the presence of the eyelid or other transitory or seasonal effects and how they contribute to the computation of the estimates. For example, when the engine is implemented as a decision tree, the related data can include the attributes defining the final class to which the user's data belongs. The related data can indicate how the estimates compare to a known range for eye pressure levels, including whether they are considered extreme or how they compare to known ranges for specific groups of users. For example, the related data can indicate the percentile of the estimates with respect to the final class to which the user belongs, or the likelihood that the user's eyes suffer from a disease. When the user attributes include those corresponding to eye diseases, the way the user's values are clustered with the given items may directly indicate whether the estimates indicate eye diseases. In addition, the server 102 can rebuild the engine every time the set of items used to build the engine is updated or according to a specific schedule.

In some embodiments, some of the features supported by the server 102 can be additionally or alternatively supported by the client device 106, and vice versa. The system can also be implemented by a single apparatus.

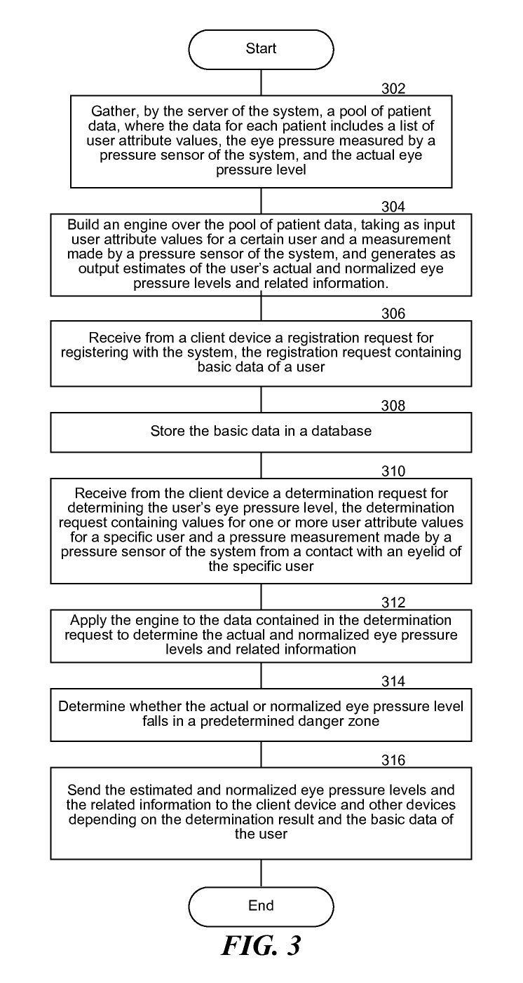

FIG. 3 illustrates an example process performed by the server for estimating eye pressure level. In some embodiments, the server first builds the engine for estimating an eye pressure level. In step 302, the server obtains a pool of patient data. Each patient data includes a list of user attribute values, eye pressure measured from a contact with an eyelid by the pressure sensor within a client device, and a value for the actual eye pressure (which can be measured by a conventional technique that requires direct eye contact). In step 304, the server builds the engine based on the pool of patient data using existing machine learning techniques, such as linear regression, decision trees, or neural networks. The server can build a two-tier engine where the first tier is aimed at eliminating the effect of an eyelid, and the second tier is aimed at reproducing the transitory and seasonable effects. Functionally, the engine takes as input a set of values for one or more user attributes for a certain user and a measurement made by the pressure sensor within a client device of the certain user and generates as output an estimate of the user's actual eye pressure level, an estimate of the user's normalized eye pressure level, and related information. Once the engine is built, the server can begin using the engine to estimate an eye pressure level. Over time, the server can also update the engine as the pool of data changes. Initially, the server allows a user to register with the system, providing basic data regarding the user. In step 306, the server receives a registration request containing the basic data from a client device of the user. The basic data can contain demographic information, medical history, family history, contact information, or various preferences. In step 308, the server stores the basic data in a database for future use.

In some embodiments, subsequently, the server can receive a determination request from the client device from time to time indicating the user's desire to determine his or her eye pressure levels. In step 310, the server receives such a determination request from the client device. The determination request contains values for the user attribute and eye pressure measured by the pressure sensor of the client device from a contact with the user's eyelid. In step 312, the server then applies the engine to the data contained in the determination request to determine the actual eye pressure level and the normalized eye pressure level. Before or as part of this application, the server may need to impute the values for some of the user attributes, based on values contained in the present determination request or previous determination requests submitted by the client device, in the basic data submitted by the client device, in the determination requests submitted by client devices of similar users, etc. The application also leads to additional information related to the actual and normalized eye pressure levels, such as how different factors or user attributes contribute to the determination of the user's eye pressure level, or how much confidence is assigned to the determination result. In step 314, the server interprets the estimates of the actual eye pressure level and the normalized eye pressure level. Specifically, the server compares the estimate of the actual or normalized eye pressure level against the general range for eye pressure levels and determines whether the estimates signal any abnormality in the eyes. In step 316, depending on the determination result, the server sends the estimates and the related information to appropriate destinations. For example, when the estimate of the normalized eye pressure level suddenly and significantly spikes or has risen to the level generally seen in glaucoma patients, the server not only sends the estimates and the related information to the client device of the user, but it also sends appropriate data to the devices of the user's eye doctor, emergency contact, or other relevant parties.

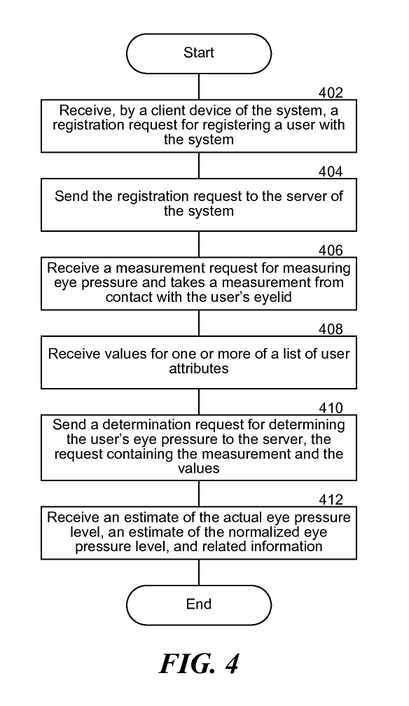

FIG. 4 illustrates an example process performed by a client device for determining an eye pressure level. Initially, the client device registers a user with the system. In step 402, the client device receives a registration request for registering a user with the system. The request can be received from a graphical user interface (GUI) displayed to the user or from a hardware interface of the client device. In step 404, the client device sends the registration request to the server, which will register the user with the system. Subsequently, the client device enables the user to determine the user's eye pressure level anywhere and anytime. In step 406, the client device receives a measurement request for measuring the eye pressure and takes a measurement using the pressure sensor. The request can be received from the GUI or the hardware interface. The client device can also automatically start the measurement process when the pressure sensor is sufficiently close to a surface. In addition, the client device can provide the user with information or feedback during the measurement process, such as a beep when an initial pressure is detected and another beep when a stable pressure level is obtained. In step 408, the client device receives values for one or more of the user attributes used to determine the actual eye pressure level by the server, such as through another GUI displayed to the user. The client device can also receive these values before taking a measurement, as described in step 406. In step 410, the client device sends a determination request to the server, including the measured eye pressure and the values for the one or more user attributes. In step 412, the client device receives from the server estimates of the user's eye pressure level and the normalized eye pressure level, together with related information regarding how the estimates are computed, what they mean, what the next steps are, etc.

It is to be appreciated that the system disclosed in the present application can also be applied to other portions of a user's body to determine the user's blood pressure level. In that case, some of the user attributes discussed above continue to apply, and more can be added related to specific portions of the body.

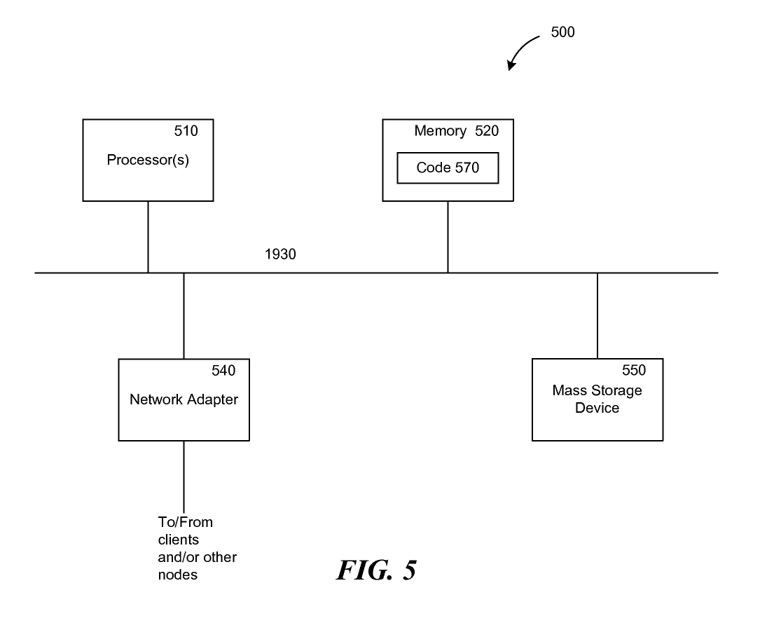

FIG. 5 contains a high-level block diagram showing an example architecture of a computer, which may represent any electronic device, such as a mobile device or a server, including any node within a cloud service as described herein, such as may implement the operations described above. The computer 500 includes one or more processors 510 and memory 520 coupled to an interconnect 530. The interconnect 530 shown in FIG. 5 is an abstraction that represents any one or more separate physical buses, point to point connections, or both connected by appropriate bridges, adapters, or controllers. The interconnect 530, therefore, may include, for example, a system bus, a Peripheral Component Interconnect (“PCI”) bus or PCI-Express bus, a HyperTransport or industry standard architecture (“ISA”) bus, a small computer system interface (“SCSI”) bus, a universal serial bus (“USB”), IIC (“I2C”) bus, or an Institute of Electrical and Electronics Engineers (“IEEE”) standard 1394 bus, also called “Firewire”.

The processor(s) 510 is/are the central processing unit (“CPU”) of the computer 500 and, thus, control the overall operation of the computer 500. In certain embodiments, the processor(s) 510 accomplish this by executing software or firmware stored in memory 520. The processor(s) 510 may be, or may include, one or more programmable general-purpose or special-purpose microprocessors, digital signal processors (“DSPs”), programmable controllers, application specific integrated circuits (“ASICs”), programmable logic devices (“PLDs”), field-programmable gate arrays (“FPGAs”), trusted platform modules (“TPMs”), or a combination of such or similar devices.

The memory 520 is or includes the main memory of the computer 500. The memory 520 represents any form of random access memory (“RAM”), read-only memory (“ROM”), flash memory, or the like, or a combination of such devices. In use, the memory 520 may contain code 570 containing instructions according to the techniques disclosed herein.

Also connected to the processor(s) 510 through the interconnect 530 are a network adapter 540 and a mass storage device 550. The network adapter 540 provides the computer 500 with the ability to communicate with remote devices over a network and may be, for example, an Ethernet adapter. The network adapter 540 may also provide the computer 500 with the ability to communicate with other computers.

The code 570 stored in memory 520 may be implemented as software and/or firmware to program the processor(s) 510 to carry out actions described above. In certain embodiments, such software or firmware may be initially provided to the computer 500 by downloading it from a remote system through the computer 500 (e.g., via network adapter 540).

### CONCLUSION

The techniques introduced herein can be implemented by, for example, programmable circuitry (e.g., one or more microprocessors) programmed with software and/or firmware, or entirely in special-purpose hardwired circuitry, or in a combination of such forms. Software or firmware for use in implementing the techniques introduced here may be stored on a machine-readable storage medium and may be executed by one or more general-purpose or special-purpose programmable microprocessors.

In addition to the above-mentioned examples, various other modifications and alterations of the invention may be made without departing from the invention. Accordingly, the above disclosure is not to be considered as limiting, and the appended claims are to be interpreted as encompassing the true spirit and the entire scope of the invention.

The various embodiments are described above with reference to flowchart illustrations and/or block diagrams of methods, apparatus (systems) and computer program products. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

A “machine-readable storage medium”, as the term is used herein, includes any mechanism that can store information in a form accessible by a machine (a machine may be, for example, a computer, network device, cellular phone, personal digital assistant (PDA), manufacturing tool, or any device with one or more processors, etc.). For example, a machine-accessible storage medium includes recordable/non-recordable media (e.g., read-only memory (ROM); random access memory (RAM); magnetic disk storage media; optical storage media; flash memory devices; etc.), etc.

These computer program instructions may also be stored in a computer-readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatuses, or other devices to cause a series of operational steps to be performed on the computer, other programmable apparatus, or other devices to produce a computer-implemented process such that the instructions that execute on the computer or other programmable apparatus provide processes for implementing the functions/acts specified in the flowchart and/or block diagram block or blocks.

The aforementioned flowchart and diagrams illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special-purpose hardware-based systems that perform the specified functions or acts or combinations of special-purpose hardware and computer instructions.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

It is to be understood that the phraseology and terminology employed herein is not to be construed as limiting and are for descriptive purposes only.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

1. A method of determining an eye pressure level, comprising:
building, by a processor, an engine from a collection of items supplied by multiple users, multiple medical professionals, or both,
wherein each item includes a value for one or more of a plurality of attributes for a corresponding user, a measurement taken by a pressure sensor of a specific type from a contact with an eyelid of the corresponding user, and an actual pressure level of an eye corresponding to the eyelid,
wherein the actual pressure level of the eye corresponding to the eyelid is produced using a technique that requires direct eye contact,
wherein the engine takes values for the plurality of attributes and measurements taken by pressure sensors of the specific type as input, and
wherein the engine produces an eye pressure estimate as output;
receiving, by the processor, a determination request for determining an eye pressure level from a mobile computing device associated with a specific user across a computer network,
wherein the determination request includes a value for at least one of the plurality of attributes for the specific user and a measurement generated by a specific pressure sensor of the specific type from a contact with an eyelid of the specific user integrated into or connected to the mobile computing device;
based on the determination request, executing, by the processor, the engine to generate an estimate of an actual eye pressure level of an eye corresponding to the eyelid of the specific user; and
sending the estimate to the mobile computing device.

2. The method of claim 1, further comprising:
receiving a registration request for registering the specific user from the mobile computing device, the registration request containing basic data of the specific user; and
storing the basic data in a database.

3. The method of claim 2, wherein the basic data includes demographic information, medical history, family history, contact information, or information sharing preferences.

4. The method of claim 1, wherein the engine is implemented as one or more of decision trees, neural networks, regression models, and other machine learning techniques.

5. The method of claim 1, wherein the plurality of attributes includes features of an eyelid and environmental or personal factors affecting eve pressure.

6. The method of claim 5,
wherein the engine produces an estimate of a normalized eye pressure level that offsets effects of the environmental or personal factors, and
wherein the engine generates the estimate of the normalized eye pressure level based on values of the features of an eyelid.

7. The method of claim 6, wherein the engine produces related information indicating a medical significance or confidence level of the estimate of the actual or normal eye pressure level.

8. The method of claim 1,
wherein the computer network is a wireless network.

9. The method of claim 1, further comprising sending the estimate to additional devices according to a medical significance of the estimate and information sharing preferences of the user.

10.-24. (canceled)

25. A method of determining eye pressure, the method comprising:
building a decision-making engine for estimating eye pressure from a collection of data sets supplied by multiple users, multiple medical professionals, or both,
wherein each data set includes values for one or more attributes of a corresponding user, a measurement taken by a pressure sensor of a certain type from a contact with an eyelid of the corresponding user, and an actual pressure level of an eye corresponding to the eyelid,
wherein the actual pressure level of the eye corresponding to the eyelid is produced using a technique that requires direct eye contact,
wherein the decision-making engine takes values associated with the one or more attributes and measurements taken by pressure sensors of the certain type as input, and
wherein the decision-making engine produces an eye pressure estimate as output;
receiving a request to determine eye pressure from a mobile computing device associated with a specific user,
wherein the request includes a value for at least one of the one or more attributes of the specific user and a measurement generated by a specific pressure sensor of the certain type that is disposed beneath a display screen of the mobile computing device,
wherein the pressure measurement is generated by the specific pressure sensor responsive to the display screen of the mobile computing device being pressed against an eyelid of the specific user;
responsive to receiving the request, executing the decision-making engine to generate an estimate of an actual eye pressure of an eye corresponding to the eyelid of the specific user; and
sending the estimate to the mobile computing device for review by the specific user.

26. The method of claim 25, wherein the mobile computing device is a cellular phone, a wearable device, or a tablet.

27. The method of claim 25, further comprising:
comparing the estimate to a threshold value; and
responsive to a determination that the estimate exceeds the threshold value, sending a notification to a medical professional or an emergency contact associated with the specific user.

28. The method of claim 1, wherein the specific pressure sensor is an air puffer.

29. The method of claim 25, wherein the multiple medical professionals include one or more eye doctors.
